(19) United States
(12) Patent Application Publication
(10) Pub. No.: US 2007/0129425 A1
(43) Pub. Date: Jun. 7, 2007

(54) Dissolution of arterial cholesterol plaques by pharmacological preparation

(76) Inventors: Filiberto Zadini, North Hills, CA (US); Giorgio Zadini, Camarillo, CA (US)

Correspondence Address: FILIBERTO ZADINI, 2237 HILLTOP LANE, CAMARILLO, CA, 93012, US

(21) Appl. No.: 11/649,062

(22) Filed: Jan. 3, 2007

Related U.S. Application Data
(63) Continuation-in-part of application No. 11/384,150, filed on Mar. 17, 2006.
Application No. 11/384,150 is a continuation-in-part of application No. 11/373,943, filed on Mar. 13, 2006.
(60) Provisional application No. 60/739,143, filed on Nov. 22, 2005.

(51) Int. Cl. A61K 31/4025; A61K 31/401; A61K 31/366; A61K 31/22

(52) U.S. Cl. 514/422; 514/423; 514/460; 514/548

(57) ABSTRACT

A biocompatible lipid solubilzer, preferably a biliary acid or salt or a biliary precursor or derivative being made bioavailable in the systemic circulation of a patient via a variety of routes of administration including topical-mucous membrane, topical-dermatological such as via a skin patch, intravenous, subcutaneous, rectal, intramuscular, intradermal, inhalatory, intrarterial, or via specialized catheter for in loco delivery of the substance, or via a subcutaneous or intravenous infusion pump, the lipid solubilizer being capable of crossing the fibrous cap of the atherosclerotic plaque to reach and dissolve the cholesterol aggregates and in general the lipidic core within the plaque. As a result of such solubilization of the lipidic core of the plaque, the solubilized cholesterol exits the plaque and enters finely dissolved into the systemic circulation leaving behind a delipidized plaque. As a result of this pharmacological action upon the atherosclerotic plaque by the biocompatible lipid solubilizer, the plaque is no longer vulnerable to rupture and arterial flow is restituted to physiological pre-plaque formation values. This effect on the lipid core of the plaque is expected to reduce and/or eliminate altogether preexisting atherosclerotic lesions and significantly reduce chances of acute and chronic ischemic events.

### RELATED CASES

This Application is the C.I.P of Applicants Patent Application entitled “Dissolution of arterial cholesterol plaques by pharmacological preparation”, application Ser. No. 11/384,150, filed on Mar. 17, 2006 which is the C.I.P of Applicants Patent Application entitled “Dissolution of arterial cholesterol plaques by pharmacological preparation”, application Ser. No. 11/373,943, filed on Mar. 13, 2006 which is the Non-Provisional Patent Application of Applicants corresponding Provisional Patent Application No. 60/739,143 entitled “Dissolution of arterial cholesterol plaques by pharmacological preparation”, filed on Nov. 22, 2005.

### FIELD OF INVENTION

This application relates to pharmacological compounds useful in the treatment of atherosclerotic plaques aiming at their dissolution.

### BACKGROUND OF THE INVENTION

Atherosclerosis is a pathological condition responsible of the highest mortality and morbidity in humans.

No known pharmacological compound has unequivocally shown in studies to effectively significantly reduce atherosclerotic lesions to the point that clinical benefits would ensue.

There are medications which act on the serum cholesterol by lowering it significantly, The effect of cholesterol lowering translates into reduced probability of new plaques formation, however, lowering of serum cholesterol does very little to the preexisting plaques.

Once an atherosclerotic plaque is formed within an artery over the years, such as coronary, cerebral, carotid, iliac, femoral, popliteal arteries, aorta and others, there is little that can be done to reduce its potential for devastating complications or make it disappear altogether and restore arterial anatomical integrity.

Although an atherosclerotic plaque is a rather complex pathological process including fat deposition, mainly cholesterol, in the intima layer of the arteries, cellular components, and a fibrotic component, the key target both in preventing formation of new plaques and in treating the preexisting plaques is the cholesterol deposition within the intima layer of the arteries. In fact, a number of controlled studies have shown that drastic reduction in blood cholesterol maintained for an adequate period of time appears to slow down progression of the plaque toward the two possible evolving paths of the plaque, one evolving path being a mere increase of the plaque size with resulting stenosis of the artery, the other evolving path being plaque disruption complicated with thrombus formation and sudden obstruction of blood flow which can lead to major events such myocardial infarction, cerebrovascular accident and death.

It appears that by removing the cholesterol and other lipids content of the plaque, the plaque may regress to the extent of reducing its size and therefore reduce the stenotic effect on the artery, and, even more importantly, to the extent of reducing or eliminating altogether the possibility of disruption of the plaque.

With respect to potential for disruption of an atherosclerotic plaque with the ominous complications that ensue as result of the disruption, there is plenty of evidence in the current medical literature that plaque susceptibility to disruption is proportional to the amount of soft lipid core of the plaque and inversely proportional to the thickness of the fibrous cap separating the lipid core from the blood. The larger the amount of lipid core of the plaque combined with a thin fibrous cap the higher the susceptibility to disruption and the higher the thrombogenicity of the disrupted plaque.

It is not illogical that attempts aimed at inducing regression of the atherosclerotic plaques or at least at reducing susceptibility to disruption have been directed to lowering the lipid content of the lipid core of the atherosclerotic plaque. A few pharmacological approaches have been attempted to reduce the lipid content of the lipid core of the atherosclerotic plaque.

The most promising pharmacological compounds presently under investigation are the Apoliprotein—A1 Milano discovered in Italy over thirty years ago by an Italian scientist named Carlo Sirtori, and, more recently found, a pharmacological compound named D-4F, which is a novel Apo A I Mimetic Peptide which acts as Apoliprotein—A1 Milano but it can be taken orally, contrary to Apoliprotein—A1 Milano which has to be administered parenterally.

Quoting Steven Nissen author of a landmark study about ApoA-1 Milano published in the Jama, Volume 290 No. 17, Nov. 2003, “the mechanisms of action of ApoA-1 Milano that result in regression of atherosclerosis are unknown but presumably are related to an increase in reverse cholesterol transport from atheromatous lesions to the serum with subsequent modification and removal by the liver.”

Both ApoA-1 Milano and D-F4 proteins act by mobilizing the cholesterol out of the plaques with a mechanism named reverse cholesterol transport, not by dissolving the cholesterol within the plaques.

Indeed none of the drugs being investigated to reduce the lipid content of the lipid core of the atherosclerotic plaque acts as detergent, as surfactant, as emulsifier, as dissolver of cholesterol aggregates or generally of the lipidic core of the atherosclerotic plaque. Applicants, in the present application, have taken a totally novel scientific approach and a novel path in the problem of reducing atherosclerotic plaque. Applicants introduce the novel concept that a cholesterol plaque can be significantly reduced and virtually eliminated by a process of emulsification of the main component of the plaque, which is the cholesterol aggregates, or any lipid content, within the plaque. Applicants propose emulsification of cholesterol plaque with a variety of emulsifiers, however, their preferred emulsifiers are compounds classified as biliary salts or acids. Biliary salts or acids are potent emulsifiers of cholesterol selected by nature to emulsify cholesterol in the intestine. Applicants have discovered and demonstrated with experiments that biliary salts or acids can also emulsify the cholesterol of the atherosclerotic plaques and actually deplete the atherosclerotic plaques of their cholesterol content when they are administered in routes that allow the biliary compound to enter the systemic circulation.

An extensive worldwide search in the Patent Office and in the medical literature has shown that this approach has never been taken before, never conceived, never disclosed, never experimented, never tested before. Applicants with their provisional patent application No. 60/739,143 entitled “Dissolution of arterial cholesterol plaques by pharmacological preparation”, filed on Nov. 22, 2005, have introduced this novel concept and with their experiments in vitro disclosed below have proven its efficacy and ultimately its usefulness.

### BRIEF SUMMARY OF THE INVENTION

In studying the physio-pathology of the atherosclerosis, Applicants have come to the conclusion that the removal of preexisting atherosclerotic plaques should entail the use of compounds capable of exhibiting two properties:
a first property consisting of being capable of dissolving the cholesterol and other lipids aggregates/deposits within the atherosclerotic plaque into such small particles or micellae, eventually even down to molecular size, to enable filtration into the blood stream of the dissolved cholesterol and other lipids through the fibrous cap which covers the cholesterol and lipids deposits in the atherosclerotic plaques;
a second property consisting of being capable of accessing the cholesterol aggregates or lipid content within the plaque by overcoming the barrier represented by the fibrous cap of the atherosclerotic plaque.

In their quest to find a compound exhibiting the first property, Applicants have focused their attention to the bile compounds responsible of solubilization of lipids during the process of digestion in the digestive system, namely the biliary salts, relying on their effectiveness in solubilizing virtually any organic lipid utilized by living beings, effectiveness which had been physiologically tested over a span of millions of years of evolution.

In animals, as in human bodies, bile salts however are confined to the digestive system, in the so called entero-hepatic circulation, and do not come in contact with arteries either of the systemic or pulmonary circulation, therefore the biliary salts in nature are prevented from displaying their benefits on atherosclerotic plaques.

Both the first and the second postulated property found confirmation in actual experiments conducted by the Applicants, experiments which will be described below in the specification section of the application.

As mentioned above, Applicants propose the use of compounds named emulsifiers or detergents or surfactants or generally lipid solvents that solubilize lipids in water in the field of the atherosclerosis.

As mentioned above, the concept of using the process of emulsification of cholesterol and of other lipids contained in atherosclerotic plaques to deplete the plaques of their cholesterol and of the other lipids contained within the plaques, as well as the use of compounds having the property of emulsifing, i.e. dissolving lipids into an acqueous phase such as blood represent an absolute novelty in the treatment of atherosclerosis.

A worldwide search in the medical and generally scientific literature and in the Patent Office has revealed no prior art referring to the use of the process of emulsification in the treatment of atherosclerotic plaques, nor to the use of compounds as emulsifiers, particularly emulsifiers which are highly water soluble while still maintain a high affinity for lipids, such as deoxycholate and, generally, biliary acids or salts.

Deoxycholate has been used widely in medicine for other purposes, precisely as an aqueous solubilizing agent of hydrophobic “liposolubil” compounds such as Amphotericin B, Diazepam, Paclitaxel, and Phosphatidylcholine.

As evidenced by the fact that, as already mentioned, there is no single reference in world medical literature or in the Patent Office of their use as plaque emulsifying/dissolving agents, no author has ever realized that deoxycholate or deoxycholic acid, usually abbreviated as DCA, or any compound of the class of substances generally named biliary acids or salts, has the capability of emulsifying the cholesterol or lipids contained in atherosclerotic plaques nor any author has demonstrated, or even postulated, that this class of compounds can cross the fibrous cap of atherosclerotic plaques to reach the cholesterol or lipids contained in the atherosclerotic plaques, in order to emulsify, i.e. liquefy, i.e. solubilize the plaques cholesterol or lipids into water and allow filtering of the emulsified cholesterol or lipids through the fibrous cap into the blood stream.

In the specific case of Phosphatidylcholine, usually abbreviated as PPC or PC, which has been used empirically as an atherosclerosis treating medication, albeit not as an emulsifier, the Deoxycholic acid which is added to the PPC, is not added as an emulsifier of cholesterol or lipids contained within the atherosclerotic plaques, but it is added, as amply documented, to the PPC exclusively for the purpose of solubilizing in water the otherwise water-insoluble phospatidylcholine.

To the date of the filing of Applicants PPA Nov. 22, 2005 and even up to the filing date of present application, Applicants have not found a single reference anywhere in the PTO/PCT or medical or generally scientific literature on the use of deoxycholic acid or any other biliary salt, primary or secondary, precursor or derivative, as direct atherosclerotic plaque dissolving agent. As clearly pointed out to the Applicants by the Chief Pharmacist of the largest Phoshatidylcohline manufacturer and supplier in USA, DCA is added to the PPC as “pharmacological necessity” i.e the necessity of solubilizing the PPC, otherwise non utilizable, as PPC is non water soluble. Reference is available.

Indeed, in the case of PPC/DCA combination, there are zero references on the use of deoxycholic acid as an antiatherogenic compound, while the emphasis is solely focused on the phosphatidylcholine as a cell membrane restoring agent. Should the DCA have ever been considered the actual active compound, it would hardly make sense to combine PPC to DCA in a 2:1 ratio formulation, which is the formulation being used in empirical attempts to treat cholesterol plaques, because the entire amount of DCA would be presumably used to dissolve PPC in water leaving no fraction of DCA, or no substantial portion of DCA, available for directly acting on the atherosclerotic plaques.

As for the phosphatidylcholine being used for treatment of high cholesterol and vascular diseases, such use was introduced by Dr. Sam Baxas at Baxamed of Switzerland a few years ago under the name of Plaquex. Plaquex is the commercial name of a pharmacological preparation, precisely a combination of PPA and DCA, in the ratio 2:1.

It is injected intravenously in patients.

In Dr. Baxas Website, www, Baxamed.com, at the date of Applicants PPA filing and at the date of the filing of the present Patent Application describes the action of PPA as follows:

“The most important effect of EPL”, an abbreviation standing for Essential PhoshoLipids, such as phosphatidylcholine and phosphatidylserine, in the respective ratio of 75% and 30%, “is its remarkable ability to reduce plaque depositions.”

The EPL is not disclosed as an emulsifying/solubilizing agent of the lipidic core of the plaque. The effect of EPL is explained solely as a cellular membrane restoring agent. The following paragraph is copied word by word from Baxamed Web Page in its entirety, not for the scientific pertinence of the paragraph, but as documentation that no mention is made of the deoxycholic acid as having any relevance at all as an ingredient acting upon the cholesterol plaques and as documentation that EPL is never mentioned to have any emulsifying/solubilizing effect on the lipidic core of the plaque. Indeed, the only ingredient that is discussed as active on atherosclerosis is the Essential Phospholipids, i.e. phosphatidylcholine and phosphatidylserine. More specifically, even in the empirically used PPC/DCA combination for atherosclerosis, there is no conception of the process of emulsification of the cholesterol and other lipids of the plaques, nor there is mention of DCA as an agent being used as an emulsifier/solubilizer of the cholesterol and other lipids of the plaques, nor, again, there is any mention of a possible emulsifying process of the cholesterol and other lipids of the plaques being induced or carried out by phosphatidylcholine or phosphatidylserine. This is the Baxamed paragraph:

“The treatment is with a mix of essential phospholipids (EPL) derived from soy beans. It is the treatment of choice for atherosclerosis—the deposit of fatty plaques in the arterial and capillary lining of the blood vessels. EPL is a natural substance, that is part of every living cell-plant cell, animal cell and human cell. The exact chemical name is phosphatidylcholine. This is a molecule made of glycerine and 2 poly-unsaturated fatty acids. It belongs to the group of Di-Ester molecules. All cell walls are mainly made out of phosphatidylcholine. 70% of a human cell wall is phosphatidylcholine and 30% is phosphatidylserin. In a watery solution, phospholipids build double layered membranes. In between the double layered phospholipid molecules structural proteins and also LDL cholesterol are inserted to help with the exchange of substances through the cell wall and to give the cell wall stability. WHY DOES EPL WORK? Damage to the cell membrane leads to LDL cholesterol being thrown out of the membrane structure, leading to elevated LDL cholesterol in the blood serum. This damage to cell walls is caused by free radicals, toxic substances and detergents that reduce the surface tension. It can also be caused by heart catheters in narrow curves ‘scratching’ the inner lining of the coronary vessels. This leads to a higher need for phosphatidylcholine. The body's own synthesis isn't enough to effect repairs. Thus scar tissue replaces the damage and plaques form inside of blood vessels. Therefore it is logical to supplement phosphatidylcholine by infusion when cell membrane damage exists. Oral supplementation is usually absorbed by the liver to repair liver damage and only minute amounts end up in other places. This is the reason oral phosphatidylcholine has little effect on blood vessels. In case of inflammation, damage to blood vessels can be stopped by phosphatidylcholine. In addition LDL cholesterol is reintegrated into the cell membrane and the serum LDL cholesterol normalizes. LDL cholesterol that has been oxidized by free radicals is bound in to micelles by phosphatidylcholine and transported to the liver where it is metabolized or excreted with gall fluid. The viscosity of the blood—the blood flow characteristics—is also improved. The main place of action by EPL is the entire capillary net. The exchange of substances such as oxygen and nutrients is improved in all tissues.

The most important effect of EPL is its remarkable ability to reduce plaque depositions in the arterial walls. It also lowers cholesterol and homocystein levels. Studies in lab animals have shown that it increases their life span by up to 36%. An important therapeutic application of the EPL treatment program is increasing an individuals ability to withstand cardiac stress. This application is valuable for the individuals who have suffered cardiac trauma, such as myocardial infarction or who are at high risk of heart trauma. Effect of EPL. EPL reduces Angina Pectoris pain and frequency of attacks EPL lowers LDL Cholesterol EPL increases HDL Cholesterol EPL improves walking distance EPL improves mental function EPL improves sexual potency EPL is useful in the treatment of patients with angina pectoris, with reduced blood flow to the brain and extremities and prophylactically in the treatment against fat embolus and strokes. EPL can be combined with Chelation treatments in severe cases. A good rule of thumb is one Chelation infusion for every two Plaquex treatments.”

End of the reported paragraph. Essentially, as a major component of cell membranes, phosphatidylcholine, is believed to be useful in the treatment of atherosclerotic plaques as a supplier of replacement material to restore cell membranes believed to be damaged in the process of atherosclerosis. Remarkably, a mention is made in the reported paragraph to the ability of phosphatidylcholine and phosphatidylserine to repair damages caused, among other factors, by detergents!

Being used as a membrane restoring agent, phosphatidylcholine in Baxamed Plaquex is not chemically optimized to act as an emulsifier of the cholesterol or of other lipids contained in the atherosclerotic plaques, although the very weak aqueous solubility of phosphatidylcholine does not make it an ideal emulsifier of cholesterol plaque. Its ability to cross the fibrous cap of the atherosclerotic plaques to exert its potential emulsifying capability upon the cholesterol and other lipids of the plaques is another property required to phosphatidylcholine to be effective as an emulsifier in atherosclerotic plaques has never been thought of, contemplated, envisioned, disclosed, not to say tested or demonstrated.

Summarizing, with the present invention, Applicants are the first to disclose the process of emulsification, i.e. water solubilization, to be applied to the cholesterol and to other lipids of the atherosclerotic plaques as a viable process to treat atherosclerotic plaques, because Applicants have discovered that certain emulsifiers are capable of crossing the fibrous cap of atherosclerotic plaques and reach the cholesterol and other lipids of the plaques, and have also discovered that when emulsified, i.e. solubilized, into water, cholesterol and other lipids contained in the plaques are capable of filtering through the fibrous cap of the atherosclerotic plaque into the blood stream.

With the present invention Applicants are the first to propose a novel and useful use of a physiological class of emulsifiers, namely the biliary acids or salts, and in general any water soluble emulsifier, in the treatment of atherosclerotic plaques.

Although, as mentioned above, in some cases biliary compounds have been used by intravenous administration in association with liposoluble medications as emulsifiers to render such medications water soluble, the amounts of biliary compound used as emulsifier for such medications were optimized to achieve the specific purpose of solubilizing the liposoluble medications in water leaving no substantial portion, or no fraction, of biliary compound available for direct pharmacological effects of the biliary compounds for instance on atherosclerotic plaques.

### OBJECTS OF THE INVENTION

It is an object of the present invention to provide a pharmacological compound capable of dissolving the lipidic core of preexisting arterial atherosclerotic plaques.

It is an object of the present invention to disclose a process of dissolution of the lipidic core of the atherosclerotic plaques consisting of emulsification of the lipidic content of the atherosclerotic plaques.

It is an object of the present invention to provide a pharmacological compound which has the ability of overcoming the barrier represented by the fibrous cap roofing the cholesterol deposits in the atherosclerotic plaques.

It is an object of the present invention to provide a pharmacological compound that solubilizes the cholesterol aggregates and other lipid aggregates within the atherosclerotic plaque to such fine particles to enable filtration of such solubilized particles through the fibrous cap of the atherosclerotic plaque into the blood stream.

It is an object of the present invention to provide a pharmacological compound that restores near physiological or physiological patency to arterial vessels obstructed by atherosclerotic plaques.

It is an object of the present invention to provide a pharmacological compound that, by removing the most critical component of an atherosclerotic plaque, i.e. the cholesterol and other lipid content of the plaque, has the ability of contributing to stabilization of the plaque, by minimizing the vulnerability of the plaque to rupture and the consequent ominous thrombus formation.

It is an object of the present invention to provide a pharmacological compound which has the potential ability of preventing the common complications of atherosclerosis such as acute coronary events and cerebrovascular accidents.

It is an object of the present invention to provide a pharmacological compound potentially useful in the treatment of peripheral vascular disease, having the potential ability of preventing ischemic limbs disease, and ultimately amputation.

It is an object of the present invention to provide a pharmacological compound which by restoring patency to the systemic and pulmonary arterial circulation to a near physiological or to a physiological level, has all the prerequisites of likely preventing and curing a number of diseases resulting from inadequate tissue perfusion due to the pathological clogging of the arterial system up to the arterioles. The compound has all the prerequisites of preventing anoxic damages to the tissues and ultimately probably preventing and in certain cases curing a myriad of pathological conditions originating from, or complicated by, the oxygen tissue deprivation, such as cardiomyopaties, heart failure, senile dementia, vascular complications from diabetes, nephrosclerosis, systemic and pulmonary hypertension, mesenteric ischemias, cerebral atherosclerosis, macular degeneration and probably the cerebral plaque of the modern era, Alzheimer disease, likely a result of anoxic chronic insults of various etiology all converging into inadequate cerebral perfusion mainly to the cognition and memory centers.

Applicants in establishing the objects of the present invention cannot obviously foresee all the implications deriving from the clearing of the obstruction to blood flow in the human arteries. Some of these objects have been disclosed, many others will be discovered following the application of the compound.

The concept of exposing the atherosclerotic plaque to a biliary compound is the core of the invention.

### FIGURES

FIG. 1 shows a skin patch for systemic administration of the pharmacological compound.

FIG. 2 is a perspective view of one of the bio-specimens, precisely a segment of an iliac artery of a pig with atherosclerotic lesions used by the applicants in their experiments.

FIG. 2A is a top view of the bio-specimen of FIG. 2 sectioned longitudinally and fully opened.

FIG. 3 shows a fixture used by the Applicants for first type of in vitro experiments with the pharmacological compound.

FIG. 3A is a detail of the apparatus of FIG. 3.

FIG. 4 shows a detail of a stage of the first type of in vitro experiments.

FIG. 4A shows a detail of a following stage of the first type of in vitro experiments.

FIG. 5 shows a fixture used by Applicants for second type of in vitro experiments with the pharmacological compound.

FIG. 6 shows a device for the administration of the pharmacological compound, precisely a specially designed intra-arterial catheter for in loco sustained administration of the substance in arteries with atherosclerotic lesions such as coronaries or carotids or popliteal arteries.

FIG. 6A is an enlarged view of the distal segment of the of the device of FIG. 6

FIG. 6B is an enlarged view of a detail of the device of FIG. 6.

### SPECIFICATIONS

The invention includes a substance or ingredient or active principle or compound or or agent or means, namely a bile acid or bile salt or bile acid or bile salt derivative or precursor administered to human subjects via routes which bypass the enterohepatic circulation in order to become bioavailable in the systemic circulation for the purpose of dissolving the lipidic core of the arterial atherosclerotic plaques to ensue decreased vulnerability of the plaque to rupture, and reduction of arterial stenosis caused by the plaque.

Any water soluble bile salt with detergent/emulsifying activity, either natural, such as Cholic acid or salt, or Chenodeoxycholic acid or salt, or Deoxycholic acid or salt, or Lithocholic acid or salt, or any synthetic biliary compound in general, alone or in combination, or any precursor or derivative of such bile acid or salt, alone or in combination, can be used, as long as it has detergent/emulsifying/surfactant/dissolving properties for the purpose of clearing the arteries of the atherosclerotic plaques and as long as it is able to penetrate the fibrous cap and access the lipidic core of the plaque.

The list below includes a great number of the known biliary acid/salts compounds.

Cholic Acids: 1,3,12-trihydroxycholanoic acid; 1,3,7,12-tetrahydroxycholanoic acid; 3beta-hydroxy-delta 5-cholenic acid; 3 beta-hydroxychol-3-en-24-oic acid; 3′-isothiocyanatobenzamidecholic acid; 3,12-dihydroxy-5-cholenoic acid; 3,4,7-trihydroxycholanoic acid; 3,6,12-trihydroxycholanoic acid; 3,7,12,23-tetrahydroxycholan-24-oic acid; 3,7,12-trihydroxy-7-methylcholanoic acid; 3,7,12-trihydroxycoprostanic acid; 3,7,23-trihydroxycholan-24-oic acid; 3,7-dihydroxy-22,23-methylene-cholan-24-oic acid (2-sulfoethyl)amide; 3-((3-cholamidopropyl)dimethylammonium)-1-propanesulfonate; 3-((3-deoxycholamidopropyl)dimethylammonium)-1-propane; 3-benzoylcholic acid; 3-hydroxy-5-cholen-24-oic acid 3-sulfate ester; 3-hydroxy-7-(hydroxyimino)cholanic acid; 3-iodocholic acid; 7,12-dihydroxy-3-(2-(glucopyranosyl)acetyl)cholan-24-oic acid; 7,12-dihydroxy-3-oxocholanic acid; allocholic acid; chapso; chol-3-en-24-oic acid; cholanic acid; Cholic Acid (which includes the Cholates: sodium cholate; methyl cholate; benzyldimethylhexadecylammonium cholate; methyl 1,3-dihydroxycholan-24-oate; and trioctylmethylammonium cholate); cholic acid glucuronide; cholyl-coenzyme A; cholyl-lysylfluorescein; cholyldiglycylhistamine; cholylhistamine; cholylhydroxamic acid; cholylsarcosine; cholyltetraglycylhistamine; ciliatocholic acid; Dehydrocholic Acid (which includes FZ 560; Gallo-Merz; Gillazym; Hepavis; Mexase; progresin Retard; and spasmocanulase); Deoxycholic Acid (which includes: 23-nordeoxycholic acid; 3,7-dioxocholanoic acid; 3-hydroxy-polydeoxycholic acid; 3-sulfodeoxycholic acid; 6-hydroxycholanoic acid; 6-methylmurideoxycholic acid; 7-ketodeoxycholic acid; 7-methyldeoxycholic acid; Chenodeoxycholic Acid; dehydrodeoxycholic acid; deoxycholyltyrosine; desoxybilianic acid; Glycodeoxycholic Acid; hyodeoxycholate-6-O-glucuronide; hyodeoxycholic acid; Taurodeoxycholic Acid; and Ursodeoxycholic Acid); Glycocholic Acid (which includes: 3-hydroxy-5-cholenoylglycine; cholylglycylhistamine; cholylglycyltyrosine; Glycodeoxycholic Acid; and sulfolithocholylglycine); hemulcholic acid; Lithocholic Acid (which includes: 12-ketolithocholic acid; 24-norlithocholic acid; 3-dehydrolithocholylglycine; 3-hydroxy-6-cholen-24-oic acid; 3-hydroxy-7,12 diketocholanoic acid; 3-hydroxy-7-methylcholanoic acid; 3-ketolithocholic acid; 3-oxochol-4-en-24-oic acid; 3-oxocholan-24-oic acid; 4-azidophenacyl lithocholate; 7-ketolithocholic acid; BRL 39924A; glycolithocholic acid; lithocholate 3-O-glucuronide; lithocholyl-N-hydroxysuccinimide; methyl lithocholate; N-carbobenzoxy-N-lithocholyl-epsilon-lysine; N-epsilon-lithocholyllysine; sulfolithocholic acid; and Taurolithocholic Acid); muricholic acid; N-(1,3,7,12-tetrahydroxycholan-24-oyl)-2-aminopropionic acid; N-(2-aminoethyl)-3,7,12-trihydroxycholan-24-amide; N-carboxymethyl)-N-(2-(bis(carboxymethyl)amino)ethyl)-3-(4-(N′-(2-((3,7,12-trihydroxycholan-24-oyl)amino)ethyl)(thioureido)phenyl)alanine; N-cholyl-2-fluoro-beta-alanine; norcholic acid; norursocholic acid; Taurocholic Acid (which includes: (N-(7-(nitrobenz-2-oxa-1,3-diazol-4-yl))-7-amino-3alpha, 12alpha-dihydroxycholan-24-oyl)-2-aminoethanesulfonate; 23-seleno-25-homotaurocholic acid; 3,12-dihydroxy-7-oxocholanoyltaurine; 3-hydroxy-7-oxocholanoyltaurine; azidobenzamidotaurocholate; hexadecyltributylammoniun taurocholate; tauro 1-hydroxycholic acid; tauro-3,7-dihydroxy-12-ketocholanoic acid; taurodehydrocholate; Taurodeoxycholic Acid; tauroglycocholic acid; Taurolithocholic Acid; tauromuricholic acid; tauronorcholic acid); tetrahydroxy-5-cholan-24-oic acid; ursocholic acid; vulpecholic acid; bile acid sulfates.

The Glycodeoxycholic Acid includes: Glycochenodeoxycholic Acid; 7-oxoglycochenodeoxycholic acid; glycochenodeoxycholate-3-sulfate; glycohyodeoxycholic acid; the Taurodeoxycholic Acid includes: tauro-7,12-dihydroxycholanic acid; Taurochenodeoxycholic Acid; taurochenodeoxycholate-3-sulfate; taurochenodeoxycholate-7-sulfate; tauroursodeoxycholic acid; taurohyodeoxycholic acid; the Ursodeoxycholic Acid includes: 23-methylursodeoxycholic acid; 24-norursodeoxycholic acid; 3,6-dihydroxy-6-methylcholanoic acid; 3,7-dihydroxy-20,22-methylenecholan-23-oic acid; 3,7-dihydroxy-22,23-methylenecholan-24-oic acid; 3,7-dihydroxy-7-ethylcholanoic acid; 3,7-dihydroxy-7-methylcholanoic acid; 3,7-dihydroxy-7-n-propylcholanoic acid; Bamet-UD2; diamminebis(ursodeoxycholate(O,O′))platinum(II); glycoursodeoxycholic acid; homoursodeoxycholic acid; HS 1030; HS 1183; isoursodeoxycholic acid; PABA-ursodeoxycholic acid; sarcosylsarcoursodeoxycholic acid; sarcoursodeoxycholic acid; ursodeoxycholate-3-sulfate; ursodeoxycholic acid 7-oleyl ester; ursodeoxycholic acid N-acetylglucosaminide; ursodeoxycholic acid-3-O-glucuronide; ursodeoxycholyl N-carboxymethylglycine; ursodeoxycholylcysteic acid; Ursometh; the Chenodeoxycholic Acid includes: 24-norchenodeoxycholic acid; 3,7-dihydroxy-12-oxocholanoic acid; 3,7-dihydroxy-24-norcholane-23-sulfonate; 3,7-dihydroxy-25-homocholane-25-sulfonate; 3,7-dihydroxychol-5-enoic acid; 3,7-dihydroxycholane-24-sulfonate; 3-glucosido-chenodeoxycholic acid; 3-oxo-7-hydroxychol-4-enoic acid; 6-ethylchenodeoxycholic acid; chenodeoxycholate sulfate conjugate; chenodeoxycholyltyrosine; Glycochenodeoxycholic Acid which includes: 7-oxoglycochenodeoxycholic acid and glycochenodeoxycholate-3-sulfate; homochenodeoxycholic acid; HS 1200; methyl 3,7-dihydroxychol-4-en-24-oate; methyl 3,7-dihydroxycholanate; N-(2-aminoethyl)-3,7-hydroxycholan-24-amide; N-chenodeoxycholyl-2-fluoro-beta-alanine; sarcochenodeoxycholic acid; Taurochenodeoxycholic Acid; taurochenodeoxycholate-3-sulfate;taurochenodeoxycholate-7-sulfate; tauroursodeoxycholic acid.

The above list is by all means not complete. It is only reported to mention instances of the class of biliary compounds, either natural as they occur in different species or synthetic. Applicants have conducted in vitro experiments which have proven the efficacy of a biliary acid in removing the lipid core of the atherosclerotic plaques from the arterial walls of mammalians.

The in vitro experiments, explained below in details, unequivocally have proven that a biliary compound when placed in contact with an atherosclerotic plaque has the ability of:
1) penetrating into the atherosclerotic plaque passing through/traversing the fibrous cap of the plaque,
2) dissolving the cholesterol aggregates within the plaque, and in general the lipidic core of the plaque, and ultimately promoting filtration of the emulsified/solubilized cholesterol and lipidic content of the plaque throughout the fibrous cap into an aqueous solution such as the blood stream leaving in situ only a virtual cavity roofed by the fibrous cap as the plaque has been emptied out of its cholesterol/lipidic content.
   First Type of in vitro Experiment:

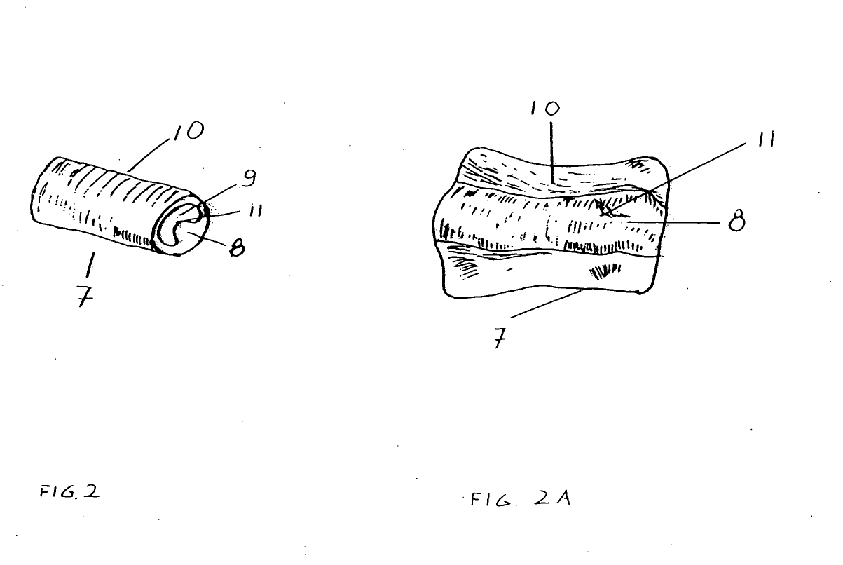

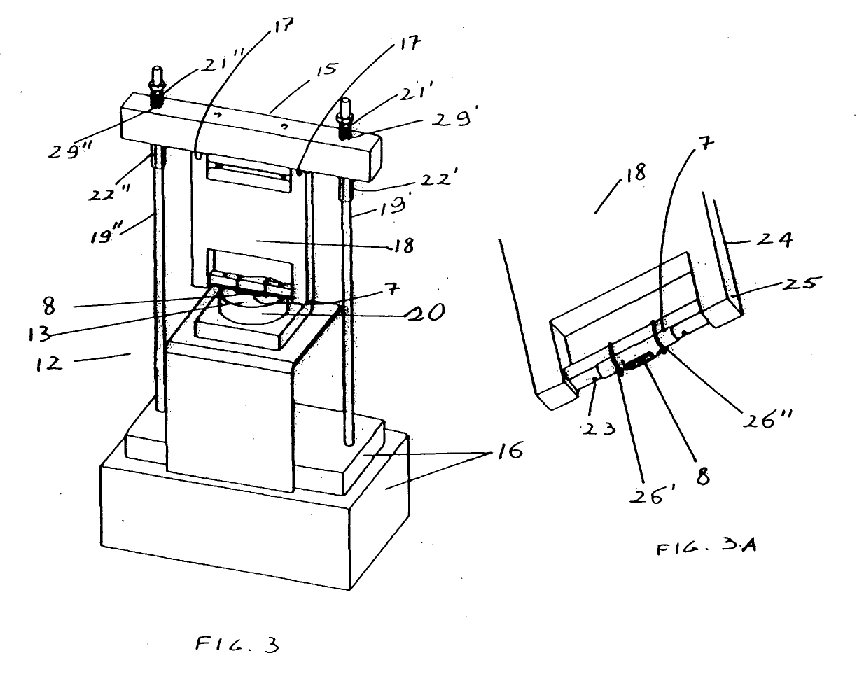

In a first type of in vitro experiment the atherosclerotic plaques of pig arteries were exposed to an aqueous solution of DCA at concentration of 50 mg./ml to test the compound in a direct plaque application model such as intracoronaric in situ delivery via intra-arterial catheter as the one disclosed below precisely in pages 31 and 32. The first type of experiments were conducted by Applicants on biospecimens of pig arteries carrying significant atherosclerotic lesions. The biospecimens were provided to the Applicants by the Pathology Department of a major US Medical College. FIG. 2 is a perspective view of iliac artery biospecimen 7. Arterial biospecimen 7 has wall 10 and lumen 9. Atherosclerotic plaque 8 protrudes from wall 10 and partially obstructs lumen 9 of artery biospecimen 7. Plaque 8 is covered by fibrous cap 11 and is contained within wall 10 of specimen 7. The major component of plaque 8 is cholesterol in form of aggregates with other lipids; the rest of the plaque contains cellular components and calcium deposits.

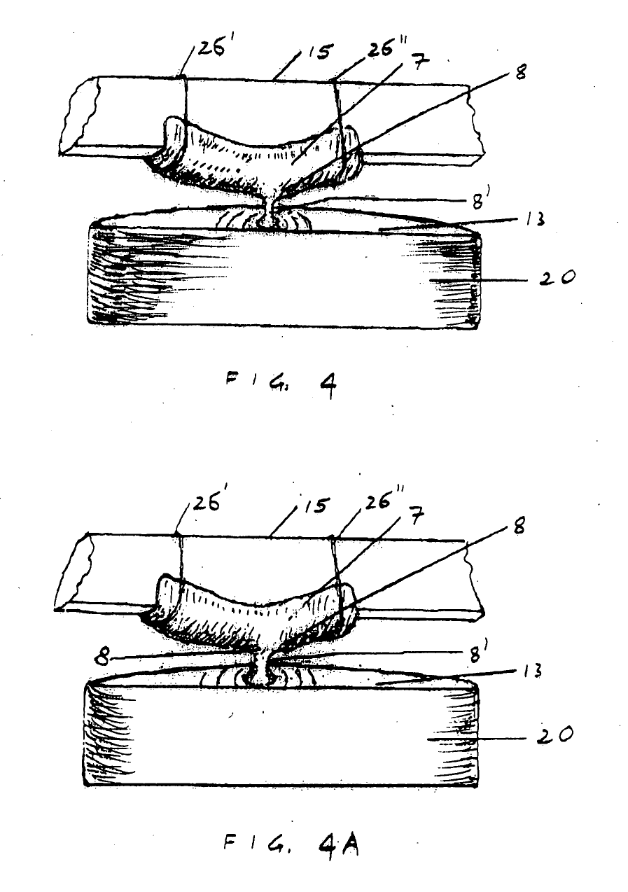

FIG. 2A shows iliac artery biospecimen 7 after being opened longitudinally. Atherosclerotic plaque 8 is recognized as a raised rib longitudinally oriented. A fixture, designated as 12 in FIG. 3 for accurate exposure of the samples to an aqueous solution of deoxycholate was constructed, consisting of rectangular frame 18 hanging via hinges 17 from a horizontal bar 15 which has vertically oriented bores 29′ and 29″ on each end slideably engaging into two parallel, vertically oriented threaded pillars 19′ and 19″ secured to a base plate 16.

Horizontal bar 15 is downwardly urged toward the base plate by springs 21′ and 21″ and retained from sliding further downward by nuts 22′ and 22″ threaded on each of the pillars 19′ and 19″. Positioning of the rectangular frame 18 along the threaded pillars 19′ and 19″ was therefore determined by positioning of height regulating nuts 22′ and 22″ along the threaded pillars 19′ and 19″.

As better shown in FIG. 3A which shows a detail of fixture 12 of FIG. 3, horizontally oriented replaceable bar 23, adapted to support specimens 7 is formed with central segment 23′ protruding downward. Bar 23 is mounted at the lower end of rectangular frame 18, being secured to lateral supports 24 of rectangular frame 18 via pins 25.

Opened biospecimen 7 is everted, wrapped around bar 23 and secured to it with ties 26′ and 26″. Atherosclerotic plaque 8 is laid in correspondence of downwardly protruding central segment 23′ of bar 23. Plaque 8 is the lowest region of biospecimen 7 mounted on horizontal bar 23 for exposure to the solution of deoxycholate 13. Container 20 filled with a solution of deoxycholate 13 is placed underneath specimen 7.

The above described spatial arrangement of the specimen is considered important to allow selective exposure of atherosclerotic plaque 8 to deoxycholate exclusively via the fibrous cap covering the plaque in order to determine permeability of the fibrous cap to the deoxycholate, and avoid exposure of the content of the plaque to the deoxycholate through the edges of the specimen.

Via rotation of the height regulating nuts 22′ and 22″ specimen 7 was lowered into the aqueous solution of deoxycholate 13 in container 20 to such a level that said lowering permitted only submersion of atherosclerotic plaque 8 which, as described above, was positioned below the rest of the specimen without allowing exposure of the raised edges of specimen 7 to the aqueous solution deoxycholate 13.

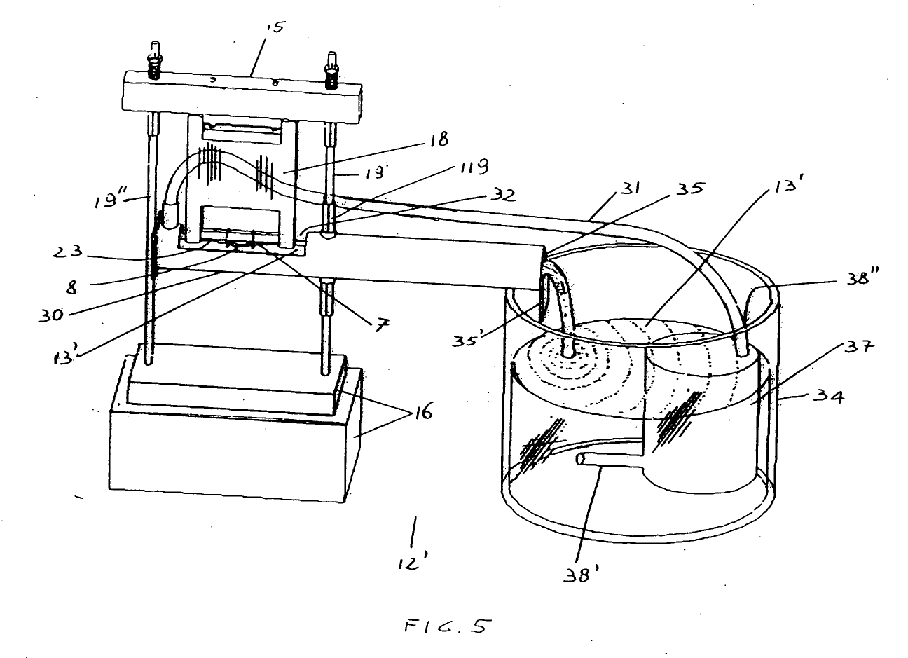

After 30 minutes of exposure of atherosclerotic plaque 7 to deoxycholate 13, via counter-rotation of the height regulating nuts 22′ and 22″ threaded on the vertical pillars 19′ and 19″, specimen 7 was lifted from the aqueous solution of deoxycholate 13. As shown on in FIG. 4 upon lifting of the specimen 7, when the lowest point of the specimen consisting of the atherosclerotic plaque 8 finally separated from the surface of the aqueous solution 13, a clear thin column 8′ of about 1-2 mm diameter, depending on the specimen, extended from the atherosclerotic plaque 8 which had been exposed to aqueous solution 13, to the surface of aqueous solution 13. Around the base of column 8′ on aqueous solution 13 the column expanded to a cone shaped base down to the level of aqueous solution 13. The clear column had a syrupy consistency and was found to be composed largely of cholesterol filtering out of the plaque through the fibrous cap covering the plaque.

The clear column of syrupy consistency completely dissolved into the aqueous solution becoming undistinguishable within the solution.

The specimen was then re-submerged in the same fashion and to the same level as the first time. After an additional 30 minutes of exposure, the specimen was lifted again, and the clear column 8′ was nearly double in diameter as shown in FIG. 4A. The process was repeated every 30 minutes and the clear column continued to increase in diameter up to approximately the third hour, then it gradually decreased until, at the fourth or fifth or sixth hour, depending on the specimen, no column was any longer visible between specimen and aqueous solution.

At macroscopic examination, the atherosclerotic plaque of the specimen being exposed to deoxycholate appeared dramatically reduced in volume, approximately between 60 to 75 percent or more in some specimen. The fibrous cap was still present, roofing a virtual cavity which prior to the experiment was largely occupied by the cholesterol aggregates. Remarkably the arterial wall appeared intact and not altered by the compound. The wall elasticity as well appeared to be well preserved. Preservation of the arterial wall integrity is expected because in physiological condition the veins of the portal system which are part of the entero-hepatic circulation do not suffer any damage from the load of biliary acids they are exposed to on daily basis. In fact, in the Review of Medical Physiology, 22^{nd} edition, FIG. 26-22, page 501, Ganong reports that the Deoxycholic acid accounts for 15% of the whole pool of human biliary acids, the remaining 85% being of cholic acid, chenodeoxycholic acid and lithocholic acid which are expected to cause the same effects, and on page 502 he reports that the total bile acids pool is of 3.5 grams and that this pool of biliary acids circulates 6 to 8 times a day from the intestine to the liver, i.e. via the veins of the portal system, and from the liver to the intestine, every day of our life.

Although no arteries are exposed, veins are, and the endothelium of the veins is similar if not identical to the endothelium of the arteries.

The specimen was then entirely bathed into the aqueous solution of deoxycholate, and after 36 hours of total exposure to deoxycholate, there were left only remnants of the atherosclerotic plaque, precisely the fibrous cap and calcium deposits.

Also after 36 hours of exposure, the arterial wall appeared intact and not altered by the compound and the wall elasticity appeared to be well preserved.

Second Type of in vitro Experiment:

In a second type of in vitro experiment, the atherosclerotic plaque of a pig artery was exposed to a continuous flow of a solution containing the compound at a very low concentration, likely a non toxic concentration, of 0.25 mg./ml, obtained diluting 1000 mg of DCA into 4 liters of Normal Saline.

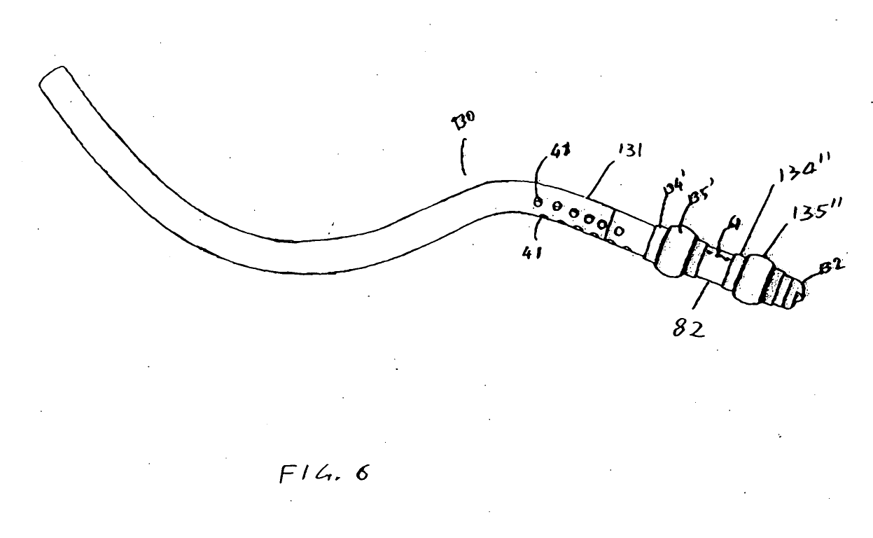

As shown in FIG. 5, experiment fixture 12′ is similar to fixture 12 of FIGS. 3 and 3A of the prior experiment except that circular container 20 is substituted by fenestrated pipe 30 for exposure of plaque 8 to the deoxycholate solution 13′. Pipe 30 mounted on pillars 19′ and 19″ is fenestrated with opening 32 for receiving bar 23 of frame 18 for exposure of plaque 8 of biospecimen 7 to circulating solution of DCA 13′. Biospecimen is designated as 7 in the description of all experiments but different specimens were naturally used in each experiment. Container 34 houses submersible pump 37. Pump 37 has an inlet port 38′ for aspiration of solution 13′ and an outlet port 38″. Solution 13′ is aspirated by pump 37 via inlet port 38′ and ejected via outlet port 38″ to circulate in mini hose 31, then in pipe 30, and it returns into container 34 via opening 35 of pipe 30.

The height of fenestrated pipe 30 is regulated by height regulating nuts 119. Barrier 35′ is slideably and sealingly mounted on end of pipe 30 at opening 35. Position of barrier 35 regulates the height of the level of solution 13 within pipe 30.

Plaque 8 of specimen 7 was clearly significantly reduced after eight days of continuous flow to the point that macroscopic examination of the plaque revealed only remnants of the plaque i.e the presence of the fibrous cap which was roofing a nearly empty plaque cavity. The cholesterol content and generally the lipidic core of plaque 8 had been dissolved by the DCA solution 13′ at a concentration of 0.25 mg/ml.

The arterial wall appeared intact and not altered by the compound and the wall elasticity appeared to be well preserved as in the prior experiment. The observations reported with the first type of experiments in respect to the expected preservation of the integrity of the arterial wall are even more valid when a low concentration of DCA is used, such as in the case of the second type of experiments.

With the above experiments Applicants have proven the following:
1. the effectiveness of the use of an emulsifier in dissolving the atherosclerotic plaque lipidic content
2. the ability of the tested emulsifier to cross the fibrous cap of the plaque to reach the lipidic content of the plaque
3. the lipid content dissolved by the tested emulsifier can filter throughout the fibrous cap of the plaque
4. the lipidic content emulsified by the tested emulsifier and filtered through the cap is completely dissolved into an aqueous solution.

In order to reach the systemic and pulmonary circulation and act upon the atherosclerotic plaques, biliary compounds or substances can be administered via many routes, except that they cannot be administered via the oral digestive route because when ingested they are absorbed by the intestine and sequestered in the entero-hepatic circulation, which keeps them away from the systemic and pulmonary circulation.

Applicants disclose below in detail one of the routes which can be used to administer the compounds, a very convenient and easy way, the topical dermatological route by the means of a skin patch.

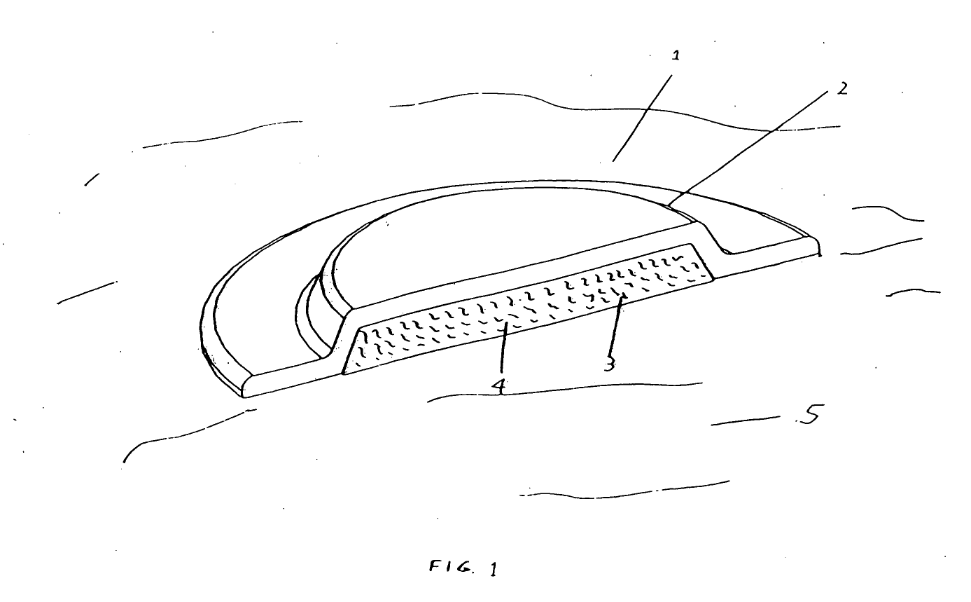

In this embodiment shown in FIG. 1 the ingredient, a biliary compound or generally an emulsifier, is delivered to the systemic circulation thru the skin in the form of a skin patch impregnated with a biliary compound or generally an emulsifier.

The skin patch generally indicated at 1 shown in FIG. 1, contains Cholic acid or Chenodeoxycholic acid or Deoxycholic acid or Lithocholic acid or any of their salts or bile salts in general, alone or in combination, or any precursor or derivative of such bile acid or salt, alone or in combination 4, such water soluble compound having detergent/emulsifing/surfactant activity.

Skin patch 1, schematically represented in FIG. 1 is composed of two layers, backing/adhesive layer 2 and reservoir layer 3, filled/impregnated with the bile compound 4 above disclosed.

Backing/adhesive substantially impermeable layer 2 serves the purpose of preventing seeping of bile compound 4 toward the exterior from patch 1 and serves mainly the purpose of permitting adhesion of patch 1 to skin 5. Reservoir layer 3, composed for instance of interwoven fabric impregnated with substance 4, in direct contact with skin 5, serves as reservoir for the delivering of substance 4 thru skin 5 into the systemic circulation.

A skin permeability enhancer along with ordinary excipents can be added to the bile acid or salt in the skin patch to facilitate the penetration and absorption of the bile acid or salt thru the skin.

The Percutaneous Chemical Enhancers which can be added can be classified as: Sulfoxides, Alcohols, Fatty acids, Fatty acid esters, Polyols, Armides Surfactants, Terpene, Alkanones Organic acids, Liposomes, Ethosomes, Cyclodextrins. Preferably, the Percutaneous Chemical Enhancers which can be used are: Ethanol, Glyceryl monoethyl ether, Monoglycerides, Isopropylmyristate, Lauryl alcohol, lauric acid, lauryl lactate, lauryl sulfate, Terpinol, Menthol, D-limonene, Beta-cyclodextrin, DMSO acronym for dimethyl sulfoxide, Polysorbates, Fatty acids e.g. oleic, N-methylpyrrolidone, Polyglycosylated glycerides, 1-Dodecylaza cycloheptan-2-one known as Azone®, Cyclopentadecalactone known as CPE-215®, Alkyl-2-(N,N-disubstituted amino)-alkanoate ester, known as NexAct®, 2-(n-nonyl)-1,3-oxolane known as SEPA®, phenyl piperazine.

The bile acid or its salt, once absorbed in the systemic circulation thru the skin, having bypassed the entheropatic circulation, will act upon the cholesterol aggregates of the atherosclerotic plaque inducing breakdown of the cholesterol aggregates of the arterial plaques, due to the well known physiological emulsifying/surfactant properties of the bile acid and or its salts.

As a result of such action by the above named substances, arterial cholesterol or atherosclerotic plaques are expected to be dissolved.

In addition to being delivered via skin patch as shown in FIG. 1, the Pharmacological Topical Preparation containing Cholic acid or Chenodeoxycholic acid or Deoxycholic acid or Lithocholic acid, or their salts alone or in combination or any precursor or derivative of such bile acid or salt alone or in combination, can be delivered into the systemic circulation via a cream means, ointment means, paste means, emulsion means, lotion means and the likes.

Physical enhancers can also be used for transdermal delivery of the above mentioned substances, such as Iontophoresis, Electroporation, Sonophoresis Thermal Poration and in general physically or chemically induced heat, Microneedles, Dermabrasion.

The bile acid or salt as disclosed above can be administered via all the other pharmacological routes of administration which bypass the enteropathic circulation:
A) Rectal, for instance in the form of a suppository.
B) Subcutaneous via injection for prompt or slow release delivery of the substance.
C) Intramuscular for prompt or slow release of the substance in a depo form.
D) Intravenous
E) Intradermal.
F) Oral mucous membrane, such as sublingual
G) Inhalation in form of inhaled microcrystals or aerosol.
H) Others, such as vaginal or intraperitoneal route

The non enterohepatic routes of administration will allow absorption of the active substance into the systemic circulation bypassing the liver. The substance will specifically target cholesterol plaques. As shown in the above experiments it will effectively promote plaque dissolution.

With regard to the sublingual route, a sweetener can be added to the compound to improve its palatability due to the notorious bitter taste of the biliary compounds. Among the intravenous routes of administration it appears particularly useful an intravenous administration via a compact, portable, ambulatory type of intravenous infusion pump that can be implanted on or applied or fastened or secured to the subject being treated, such as the Medtronic MiniMed Insulin pump.

A special and effective route of administration is the Intra-Arterial route i.e. the delivering of an emulsifying compound intra-arterially or via the use of a specialized intra-arterial catheter for a sustained contact of the substance in loco, i.e directly on to the atherosclerotic plaque and avoidance of dispersion of the substance in the systemic circulation, for treatment of identified coronary artery or peripheral arteries atherosclerotic lesions.

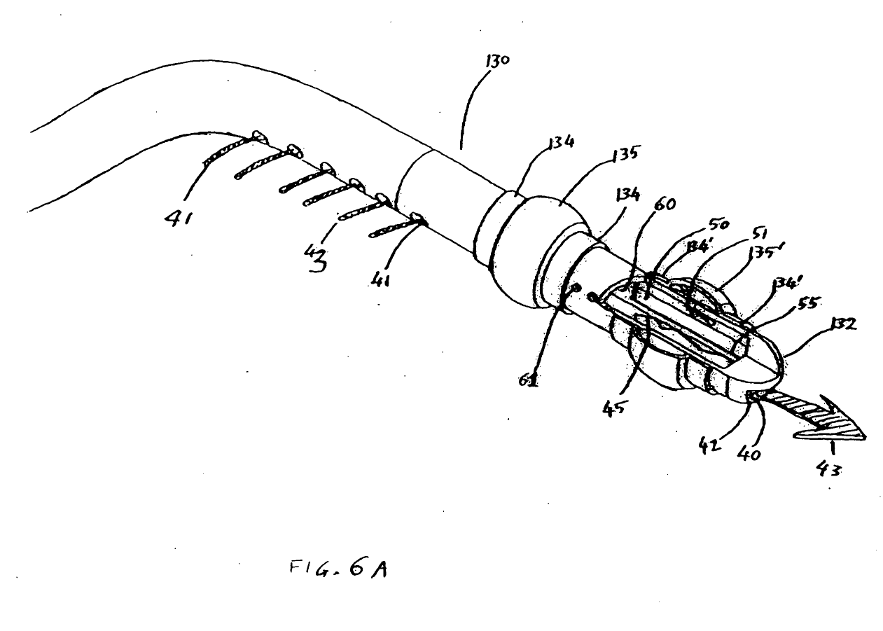

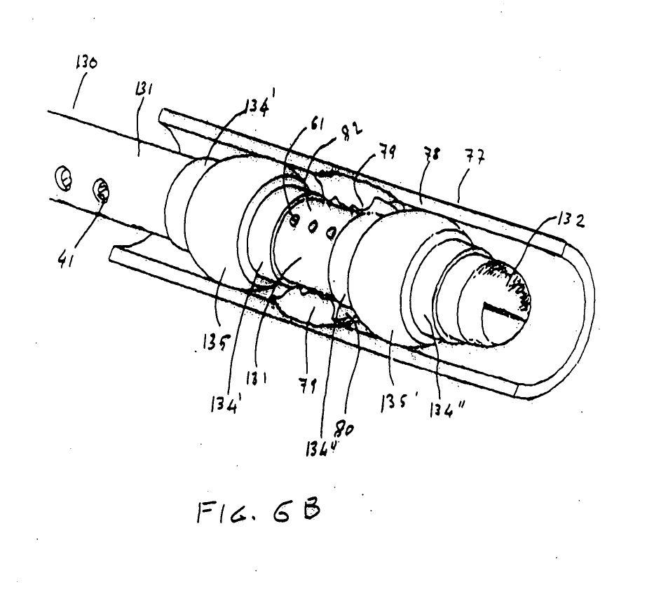

As shown in FIGS. 6, 6A and 6B, catheter 130 is composed of tubular body 131 having distally tip 132, and two generally donut shaped balloons or expandable members, distal balloon, 135″ sealingly connected to tubular body 131 of catheter 130 via sleeves 134″ and a proximal balloon 135′ sealingly connected to tubular body 131 of catheter 130 via sleeve 134′. As better shown in FIG. 6B, balloons 135′ and 135″ are spaced from each other to leave segment 82 of tubular body 131 exposed. As better shown in FIG. 6A, tubular body 131 of catheter 130 has three longitudinal compartments: compartment 40 for passage of blood 43 from inlet openings 41 to outlet openings 42 located at tip 132. This compartment is obliterated proximally to the most proximal inlet opening 41. Septum 45 separates compartment 40 from the other two compartments 50 and 60. Compartment 50 is separated from compartment 60 by septum 55 and is in flow communication with the inside of balloons 135′ and 135″ to allow inflation/deflation of balloons 135′ and 135″. As best shown in FIG. 6B, compartment 60 has openings 61 to allow compound to enter space 80, delimited distally by inflated balloon 135″, proximally by inflated balloons 135′, medially by tubular body 131 of catheter 130 and laterally by the arterial wall 78 of artery 77, which in FIG. 6B is shown longitudinally cross sectioned. Balloons 135′ and 135″ are inflated to a degree to seal space 80 from the remaining segments of artery 77.

In use tip 132 of catheter 130, as better shown in FIG. 6B, is passed in the arterial lumen beyond atherosclerotic plaque 79 of arterial wall 78 of artery 77 so as to align exposed segment 82 of tubular body 131 with atherosclerotic plaque 79. Compound is introduced into compartment 60 at the proximal end of catheter 130, to fill space 80 in suitable concentration and for an extended period of time to exert its full dissolving effect on atherosclerotic plaque 79 of arterial wall 78 of artery 77. The compound can then drained from the proximal end of compartment 60, and after balloon deflation, the catheter is removed from the artery.

The above description of catheter 130 is purely illustrative of a method for direct application of the compound on the lesioned arteries where the compound can be applied at high concentration on the arterial wall and sealed off from the arterial blood which is bypassed within the artery to avoid dispersion of the compound in the blood stream and to maximize the effect of the compound on the atherosclerotic plaques. Other known types of catheters having two discrete balloons or a dog bone shaped balloon can be used for drug delivery applications, to seal off the precise area that requires treatment. Additional intracoronary or generally intra-arterial drug delivery catheters can be used for such purpose, with different designs, such as the Dispatch by SciMed, which is multichamber autoperfusion balloon catheter, or the Channel Balloon Catheter by Boston Scientific, a local drug-delivery catheter that has the dual capability of high-pressure lesion dilation and low-pressure drug infusion.

Biliary compounds can also be chemically manipulated and designed in such a way that they are not captured by the liver in any significant amount to be sequestered into the entero-hepatic circulation once introduced into the body by any route including the oral-digestive route. The use of these types of compounds makes oral administration possible even with biliary compounds, expanding even further the possibilities of the disclosed treatment of atherosclerosis.

An interesting compound among the biliary acids is the hyodeoxycholic acid. As reported by Sacquet E. et al. in their article Intestinal absorption, excretion, and biotransformation of hyodeoxycholic acid in man, Journal of Lipid Research, Vol 24, 604-613, 1983, once it reaches the liver through the portal venous system after absorption by the intestinal mucosa, the hyodeoxycholic acid largely escapes, in healthy humans, the enterohepatic circulation entering the systemic circulation to be excreted through the kidneys in the urine in a very significant amount. It appears that the hyodeoxycholic acid escapes the enterohepatic circulation after having undergone a process of glucuronidation by the hepatic cell. The Applicants believe that this peculiarity of the hyodeoxycholic acid to enter the systemic circulation in theory could be exploited to directly emulsify/dissolve the lipid core of atherosclerotic plaques. Another advantage of the hyodeoxycholic acid is that it can be administered via oral-intestinal route. Sehayek E. et al. in their article Hyodeoxycholic acid efficiently suppresses atherosclerosis formation and plasma cholesterol levels in mice, Journal of Lipid Research, Vol. 42, 1250-1256, August 2001 report that the hyodeoxycholic acid efficiently suppresses dietary cholesterol absorption, depletes the liver content of cholesterol and cholesteryl esters, reaches the systemic circulation and undergoes urinary excretion, stimulates liver cholesterol biosynthesis, decreases plasma cholesterol levels of atherogenic lipoproteins, decreases atherosclerosis formation, while it does not promote intestinal tumorigenesis. The effect on suppressing atherosclerotic plaques is noted by the Authors to be mainly a result of the plasma cholesterol decrease induced by this acid and partially a result of other postulated plasma cholesterol independent reasons, but there is no mention in any section of the article of hypotheses that the hyodeoxycholic acid might emulsify/dissolve the cholesterol aggregates and generally the lipidic core of the atherosclerotic plaque as it does emulsify/dissolve cholesterol aggregates in the intestine. Indeed, at the time Sehayek's article was written and prior to the filing date of Applicants' PPA No. 60/739,143 filed Nov. 22 2005, there has been no notion in the medical literature that at least one type of biliary acid, the deoxycholic acid, is capable of filtering through the fibrous cap of the atherosclerotic plaque and reach the lipidic core of the plaques to emulsify/dissolve it; therefore, in absence of comparable testing for the hyodeoxycholic acid, no hypothesis on the likelihood of the hyodeoxycholic acid to cross the fibrous cap could be formulated on scientific ground. Moreover, in their article, as pointed out above, Sehayek E. et al. do not use the hyodeoxycholic acid as an emulsifier of atherosclerotic plaque nor optimize it as an emulsifier of atherosclerotic plaque.

In any event, the ability of the hyodeoxycholic acid to cross the fibrous cap of atherosclerotic plaques, and the ability of the hyodeoxycholic acid of emulsifying/dissolving the cholesterol aggregates and generally the lipidic core of the atherosclerotic plaques has not yet been established.

As a matter of fact, there is no demonstration in the above cited article, nor a conclusion nor even a hypothesis that existing atherosclerotic plaques are or can be suppressed or reduced or treated with hyodeoxycholic acid. Even when the Authors attempt to explain a reduction of atherogenesis in the animals treated with hyodeoxycholic acid which is greater than it would be expected with the sole plasma cholesterol lowering effect induced by hyodeoxycholic acid, and consequently postulate an effect of hyodeoxycholic acid on atherogenesis through mechanisms other than simple lowering of plasma cholesterol, the Authors unequivocally refer to such mechanisms as mechanisms preventing atherogenesis, not to mechanisms suppressing or reducing pre-existing atherosclerotic plaques.

The Authors indeed postulate, using their own words, that it is possible that the capacity of hyodeoxycholic acid to reach the systemic circulation may have a direct effect on the arterial wall and its cholesterol-independent effects, i.e. stimulation of hydroxymethylglutaryl-CoA reductase, HMGR, may affect atherogenesis through lipoprotein-independent mechanisms. This statement admittedly refers to an effect on atherogenesis, i.e. on atherosclerotic formation, not to an effect on already formed, pre-existing plaques. Moreover, although in the cited article language such as “decrease” of atherosclerotic aortic area is used, this language is not referred to a reduction in area of an existing plaque, but simply is referred to a smaller size of atherosclerotic plaques observed in animals being preventively treated with hyodeoxycholic acid in comparison of the control group animals which are not preventively treated with hyodeoxycholic acid. The cited study, as it was designed, involved the use of hyodeoxycholic acid at the very beginning of the cholesterol diet and continued throughout the whole period of cholesterol diet, being the purpose of the study to demonstrate inhibition of atherogenesis through reduced absorption of cholesterol induced by addition of hyodeoxycholic acid to the diet. On the contrary, a study whose purpose is to demonstrate suppression of pre-existing plaques should be structured in a way that animals are firstly fed with a high cholesterol content diet for a period of time sufficient to induce formation of atherosclerotic plaques and then, and only then, the compound is used to evaluate its ability to suppress pre-existing plaques.

The above discussion wants to stress the point that in the cited article by Sehayek et Al. there is no conception on using hyodeoxycholic acid to treat pre-existing atherosclerotic plaques and that the use of biliary acids and in general of water soluble emulsifiers to treat existing atherosclerotic plaques, as well as their emulsifying mechanism of action, is a novelty introduced by the Authors of the present invention.

Biliary compounds being designed to enter the systemic circulation through oral-digestive route of administration can be associated with intestinal absorption enhancers so that their bioavailability in the systemic circulation is maximized. The absorption of hyodeoxycholic acid or its salts, which already have the unique capability among the biliary compounds of escaping, in large percentage, the enterohepatic circulation to enter the systemic circulation, can also be enhanced via the use of intestinal absorption enhancers so as to further increase its bioavailability in the systemic circulation.

Some of the intestinal absorption enhancers which can be used are sodium glycocholate, sodium taurocholate, EDTA, sodium deoxycholate, sodium salicylate, sodium caprate, diethyl maleate, N-lauryl-beta-D-maltopyranoside, linoleic acid polyoxyethylated, tartaric acid, sodium dodecyl sulphate, p-t-octyl phenol polyoxyethylene-9.9 known as Triton X-100, Alkylglycosides such as: hexylglucoside, hexylmaltoside, heptylglucoside, octylglucoside, octylmaltoside, nonylglucoside, nonylmaltoside, decylglucoside, decylmaltoside, dodecylmaltoside, tetradecylmaltoside, dodecylglucoside, and tridecylmaltoside, and mucolytic agents such as N-Acetylcysteine and Chitosan.

For all the biliary compounds which are absorbed through the intestine and escape the enterohepatic circulation entering into the systemic circulation, Applicants also propose the use of the already available technology consisting of slow release/controlled release/long acting pharmacological preparations. Such technology includes the use of the microencapulation process, enteric drug coating technology or the use of cyclodextrin as drug vehicle.

A final consideration will follow on the relative theoretical efficacy of the various biliary compounds, as it can be reasonably predicted by their chemical characteristics.

Since intestinal absorption of cholesterol occurs as a result of conversion of the oil phase of cholesterol into the micellar phase of cholesterol which, in form of micellae, is phapocitated, therefore absorbed, by the enterocytes, biliary compounds have been classified according to their efficiency in creating micellae from the oil phase of cholesterol. Biliary compounds which have been recognized to be highly efficient in creating micellae have been consequently viewed as facilitators of intestinal absorption of cholesterol, while biliary compounds which have been found to be less efficient in creating micellae have been viewed as inhibitors of intestinal absorption of cholesterol. The biliary compounds which exhibit greater efficiency in creating micellae, as for instance the deoxycholic acid, were found to be prevalently hydrophobic, while the biliary compounds which exhibit less efficiency in creating micellae, such as for instance hyodeoxycholic acid, ursodeoxycholic acid, dehydrocholic acid, etc., were found to be prevalently hydrophilic.

This subdivision of biliary compounds in hydrophobic and hydrophilic compounds has turned out to be useful in predicting the usefulness of a biliary compound as an inhibitor or a facilitator of intestinal cholesterol absorption just on the basis of the prevalence of hydrophilic or hydrophobic groups in the compound molecule.

Using the same criteria in selecting biliary compounds based on their capability of creating micellae out of the oil phase of cholesterol, it could be reasonably predicted that highly hydrophobic biliary compounds, such as the deoxycholic acid, could be even a more promising choice in dissolving cholesterol of atherosclerotic plaques than prevalently hydrophilic biliary compounds such as hyodeoxycholic acid, ursodeoxycholic acid, and dehydrocholic acid.

A combination of hydrophobic and hydrophilic biliary compound could also maximize solubilization of cholesterol and diffusion of cholesterol in a water phase such as blood. Applicants, throughout this application, have proposed the use of compounds named emulsifiers or detergents or surfactants or generally lipid solvents/solubilizers that solubilize lipids, particularly cholesterol, in water to use them in the field of the atherosclerosis for the treatment of atherosclerosis. While Applicants have indicated preference for physiological emulsifiers as biliary acids or their salts, including their precursors and derivatives, Applicants have also proposed the use of other suitable emulsifiers that do not belonging to the biliary salts/acids class.

For sake of clarity, Applicants disclose below biological/biocompatible compounds having the properties of being capable of dissolving lipids, and cholesterol in particular, of atherosclerotic plaques, into physiological fluids. These solubilizers of atherosclerotic plaques lipids can be variously administered via the oral route of administration, the transdermal route, the parenteral, i.e intradermal, subcutaneous, intravenous, intramuscular, route of administration, the mucous membrane route, such as the oral, sublingual, rectal, vaginal, or the inhalatory or the intraperitoneal route of administration. Such compounds, which are biological/biocompatible compounds, which are biological/biocompatible detergents, can be classified according to several criteria.

Detergents Classified According to Structure:

Alkyl glycosides, which include: n-nonyl-□-D-glucopyranoside, n-octyl-□-D-glucopyranoside, n-heptyl-□-D-glucopyranoside, n-hexyl-□-D-glucopyranoside, dodecyl-□-D-maltoside, decyl-□-D-maltoside, octyl-□-Dthioglucopyranoside, and others
Bile acids, which include a very large number of compounds listed elsewhere in this application.
Glucamides, which include: MEGA-10, MEGA-9, MEGA-8, Deoxy Big CHAP, Big CHAP, and others
Polyoxyethylenes, monodisperse and polydisperse which include: reduced TRITON® X-100, reduced TRITON® X-114, TRITON® X-100, NP-40, TRITON® X-114, GENAPOL® X-080, GENAPOL® X-100, C12E8, C12E9, THESIT®, LUBROL® PX, GENAPOL® C-100, BRIJ® 35, PLURONIC® F-127®, (laurate), TWEEN® 20 (oleate), TWEEN® 80, and others
Zwittergents, which include: EMPIGEN BB® (n-dodecyl-N,Ndimethylglycine), ZWITTERGENT® 3-08, ZWITTERGENT® 3-10, ZWITTERGENT® 3-12, ZWITTERGENT® 3-14, ZWITTERGENT® 3-16, CHAPS, CHAPSO, and others
   Detergent Classified According to Electric Charges:
   Ionic Detergents, which Include:
BATC
Cetyltrimethylammonium Bromide (CTAB), Molecular Biology Grade
Chenodeoxycholic Acid, Free Acid
Chenodeoxycholic Acid, Sodium Salt
Cholic Acid, Sodium Salt
Cholic Acid, Sodium Salt, ULTROL® Grade
Deoxycholic Acid, Sodium Salt
Deoxycholic Acid, Sodium Salt, ULTROL® Grade
7a, 12a-Dihydroxy-5β-cholanic Acid
Glycholic Acid, Sodium Salt
Glycodeoxycholic Acid, Sodium Salt
Lauroylsarcosine, Sodium Salt
Sodium n-Dodecyl Sulfate (SDS)
Sodium n-Dodecyl Sulfate (SDS), High Purity
Sodium n-Dodecyl Sulfate (SDS), Molecular Biology Grade
Sodium n-Dodecyl Sulfate (SDS), 30% Solution
Taurochenodeoxycholic Acid, Sodium Salt
Taurocholic Acid, Sodium Salt
Taurocholic Acid, Sodium Salt, ULTROL® Grade
Taurodehydrocholic Acid, Sodium Salt
Taurodeoxycholic Acid, Sodium Salt
Taurolithocholic Acid, Sodium Salt
Tauroursodeoxycholic Acid, Sodium Salt
TOPPS
   Non-Ionic Detergents:
APO-10
APO-12
Big CHAP
Big CHAP, Deoxy
BRIJ® 35, PROTEIN GRADE® Detergent, 30% Solution
BRIJ® 35, PROTEIN GRADE® Detergent, 10% Solution, Sterile-Filtered
C12E6
C12E8
C12E9
Cyclohexyl-n-ethyl-β-D-maltoside, ULTROL® Grade
Cyclohexyl-n-hexyl-β-D-maltoside, ULTROL® Grade
Cyclohexyl-n-methyl-β-D-maltoside, ULTROL® Grade
n-Decanoylsucrose
n-Decyl-β-D-maltopyranoside, ULTROL® Grade 252718
n-Decyl-β-D-thiomaltoside, ULTROL® Grade
Digitonin, High Purity
Diaitonin, Alcohol-Soluble, High Purity
n-Dodecanoylsucrose 324374
n-Dodecyl-β-D-glucopyranoside 324355
ELUGENT™ Detergent, 50% Solution
GENAPOL® C-100, PROTEIN GRADE® Detergent, 10% Solution
GENAPOL® X-80, PROTEIN GRADE® Detergent, 10% Solution
GENAPOL® X-100, PROTEIN GRADE® Detergent, 10% Solution
n-Heptyl-β-D-glucopyranoside
n-Heptyl-β-D-thioglucopyranoside, ULTROL® Grade, 10% Solution
n-Hexyl-β-D-glucopyranoside
MEGA-8, ULTROL® Grade
MEGA-9, ULTROL® Grade
MEGA-10, ULTROL® Grade
n-Nonyl-β-D-glucopyranoside
NP-40, PROTEIN GRADE® Detergent, 10% Solution
n-Octanoyl-β-D-glucosylamine (NOGA)
n-Octanoylsucrose
n-Octyl-β-D-glucopyranoside
n-Octyl-β-D-glucopyranoside, ULTROL® Grade
n-Octyl-β-D-maltopyranoside
n-Octyl-β-D-thioglycopyranoside, ULTROL® Grade
PLURONIC® F-127, PROTEIN GRADE® Detergent, 10% Solution
TRITON® X-100
TRITON® X-100, PROTEIN GRADE® Detergent, 10% Solution
TRITON® X-100, Molecular Biology Grade
TRITON® X-100, Hydrogenated
TRITON® X-100, Hydrogenated, PROTEIN GRADE® Detergent, 10% Solution
TRITON® X-114, PROTEIN GRADE® Detergent, 10% Solution
TWEEN® 20
TWEEN® 20, Molecular Biology Grade
TWEEN® 20, PROTEIN GRADE® Detergent, 10% Solution
TWEEN® 80, PROTEIN GRADE® Detergent, 10% Solution
n-Undecyl-β-D-maltoside, ULTROL® Grade
   Zwitterionic Detergents:
ASB-14
ASB-16
CHAPS
CHAPSO
DDAMB
DDAMU
EMPIGEN BB® Detergent, 30% Solution
Lauryldimethylamine Oxide (LDAO), 30% Solution
ZWITTERGENT® 3-08 Detergent
ZWITTERGENT® 3-10 Detergent
ZWITTERGENT® 3-12 Detergent
ZWITTERGENT® 3-14 Detergent
ZWITTERGENT® 3-16 Detergent

Of particular interest to Applicants are: Pluronic compounds such as F68 i.e. Polaxamer 188; Tween 80 i.e. Polysorbate 80; Triton X 100; Methyl-Butyl Ether known as MBTE; Ethylpropionate known as EP; Sorbitol Anydride Monostearate known as Span; Sorbitan compounds; taurodihydrofusidate, and d-limonene, which can be considered as an organics solvent belonging to the terpenes. Organic solvents are indeed lipid solubilizers, but they are also generally toxic. Among a few other exceptions besides the ones cited above, are certain terpenes, which are usually extracted from essential oils of plants, such as the mentioned d-limonene, and certain terpenoids, also of plant origins, as for instance terpenoid constituents of Ginkgo biloba extract:

The biliary compounds and generally the biological/biocompatible emulsifying compounds, and the biological/biocompatible lipid solubilizers/solvents, such as the d-limonene, reported above, can be used alone via the routes disclosed above or in combination with the following compounds:
1) Statins with the purpose of clearing the blood from the expected transitory cholesterol increase resulting from the lipidic dissolution of the atherosclerotic plaques induced by the emulsifying compounds object of this disclosure, to impede new plaque formation achieved by the action of the statins which effectively lower serum cholesterol.
2) EDTA with the purpose of removing the calcium deposits frequently present within the atherosclerotic plaques.
3) Lipase to add a lipolytic activity to the emulsifying activity of the compound possibly in a synergistic fashion.
4) Collagenase for the purpose of enhancing the permeability the fibrous cap of the atherosclerotic plaque and accelerating and/or facilitating and/or enhancing the penetration of DCA into the plaque. Due to its fibrolytic properties, Collagenase will act upon the fibrotic component of the plaque aiding in its dissolution.
5) Hematoporfyrins which have shown to selectively accumulate within atherosclerotic plaques in a study once administered intravenously. The complex biliary compound or generally an emulsifier with hematoporfyrins would enhance in loco delivery of the complex into the atherosclerotic plaque by selective localization and accumulation of the complex in the atherosclerotic plaques.

In this patent application Applicants have disclosed beneficial effects on atherosclerosis deriving from the delipidizing properties of biliary acids and/or other lipid solubilizers and biocompatible/biological detergents on lipidic deposits within arterial walls. As at least one of the detergents/lipids solubilizers proposed in this application has been demonstrated to be capable of crossing the fibrous cap of atherosclerotic plaques, which represents a pathological development arising from endothelial cells, it is not unlikely that the same compounds being made bioavailable in the systemic circulation could also cross the endothelial cells of capillaries to promote delipidization of subcutaneous fat deposits and, by promoting delipidization of subcutaneous fat deposits, produce beneficial reducing effects on obesity.

What we claim is:

1. A treatment for atherosclerosis, a pathological process affecting systemic circulation and characterized by the presence of atherosclerotic plaques which have an atheromatous lipidic core component mainly consisting of cholesterol aggregates and a sclerotic fibrous cap component covering the lipidic core, comprising the use of:
a pharmacological compound having a property of being a solubilizer of the lipidic core of the atherosclerotic plaque,
said solubilizer of the lipidic core of the plaque being made bioavailable in the systemic circulation and made bioavailable to act as a lipid solubilizer upon said atherosclerotic plaques,
said solubilizer of the lipidic core of the plaque having also a property of entering the atherosclerotic plaques from said systemic circulation, so as to dissolve the lipidic core of the plaque and cause depletion of the lipidic core of the plaque though the fibrous cap.

2. The lipid solubilizer of claim 1, wherein said lipid solubilizer is ionic detergent.

3. The lipid solubilizer of claim 1, wherein said lipid solubilizer is non-ionic detergent.

4. The lipid solubilizer of claim 1, wherein said lipid solubilizer is zwitterionic detergent.

5. The lipid solubilizer of claim 1, wherein said lipid solubilizer is a biliary acid or salt.

6. The lipid solubilizer of claim 1, wherein said lipid solubilizer is a biliary compound.

7. The lipid solubilizer of claim 1 being administered via an oral route.

8. The biocompatible lipid solubilizer of claim 1 being administered via a parenteral route.

9. The biocompatible lipid solubilizer of claim 1 being administered via transdermal -oute.

10. The treatment of claim 1, wherein said lipid solubilizer is being introduced into the human body via a catheter for in situ delivery of said pharmacological compound for sustained contact of said pharmacological compound directly on to the atherosclerotic plaque.

11. The lipid solubilizer of claim 10 being a Triton compound.

12. The treatment of claim 1 further comprising a statin.

13. The treatment of claim 1 further comprising a lipase to digest fatty complexes within said atherosclerotic plaques.

14. The treatment of claim 1 further comprising EDTA to reduce calcifications within said atherosclerotic plaques.

15. The treatment of claim 1 further comprising a collagenase to enhance penetration of said lipid solubilizer through the fibrous cap of the plaque and to act upon a fibrotic plaque component ultimately enhancing the effects of said lipid solubilizer upon the plaque.

16. The treatment of claim 1 further comprising hematoporfyrins being associated with said lipid solubilizer to enhance concentration of said lipid solubilizer within said atherosclerotic plaques.

17. The lipid solubilizer of claim 7 further comprising an intestinal absorption enhancer to enhance its absorption thru an intestinal mucosa.

18. The biocompatible lipid solubilizer of claim 8 being introduced into the human body via a compact, ambulatory infusion pump.

19. The lipid solubilizer of claim 10 being a biocompatible organic solvent.

20. The treatment of claim 10 further comprising a collagenase to enhance penetration of said lipid solubilizer through the fibrous cap of the plaque and to act upon a fibrotic plaque component ultimately enhancing the effects of said lipid solubilizer upon the plaque.
